(19) United States
(12) Patent Application Publication
(10) Pub. No.: US 2016/0113563 A1
(43) Pub. Date: Apr. 28, 2016

(54) TESTICULAR TORSION SENSING APPARATUS AND METHOD THEREOF

(71) Applicant: HSIAO-WEN CHEN, TAIPEI (TW)

(72) Inventor: HSIAO-WEN CHEN, TAIPEI (TW)

(21) Appl. No.: 14/620,266

(22) Filed: Feb. 12, 2015

(30) Foreign Application Priority Data
Oct. 23, 2014 (TW) 103136583

(51) Int. Cl. A61B 5/00; A61B 5/11; A61B 5/026; A61B 5/1455; A61B 5/0205

(52) U.S. Cl. CPC A61B 5/4387; A61B 5/14552; A61B 5/02055; A61B 5/0261; A61B 5/742; A61B 5/7282; A61B 5/7246; A61B 5/1123; A61B 5/746; A61B 5/1116

(57) ABSTRACT

The present invention provides an apparatus for sensing a status of testicular torsion and a method thereof, and is related to technical fields of measuring human pulse, heart rate, blood pressure, or blood flow, for sensing the status of a test area in a user. The apparatus comprises a sensor configured for projecting a specific wavelength light source to the test area and receiving a reflected light from the test area, for obtaining a pulse information and an oxygen saturation information on the test area; a comparing unit configured for determining a status of blood flow in the test area and a corresponding status of testicular torsion, according to the pulse information and the oxygen saturation information; and a display unit for displaying the testicular torsion status.

### CROSS REFERENCE

The present invention claims the benefit of Republic of China application TW 103136583, filed on Oct. 23, 2014.

### BACKGROUND OF THE INVENTION

1. Field of Invention

The present invention relates to a sensing apparatus and a method thereof, and especially relates to an apparatus for sensing a status of testicular torsion and a method thereof. The status is sensed by projecting a specific wavelength light source to a topical area under test (e.g. over a testicle) in human body, which is non-invasive, with measuring the proportion between the light absorbed by the test area and the reflected light from the test area. In order to accurately determine the status of blood flow in the test area and a corresponding status of testicular torsion, the information of arterial pulsation and an arterial oxygenated saturation are yielded through the ratio of red light and infrared red light.

2. Description of Related Art

Conventional techniques for sensing testicular torsion, such as using vascular visualizer or Doppler ultrasonography, which is a non-invasive type of sensing technique, are common sensing techniques known. However, these techniques, although non-invasive, need various equipment and consumable materials which are expensive and difficult to obtain, as well as not high accurate diagnostic rate, which are resulting in limitation of using. Other conventional sensing techniques, such as scrotal exploration surgery, which is an invasive type of procedure and which will cause more injuries and side-effects for the subject patients, and are therefore bearing with some dilemma.

In view of these above-mentioned drawbacks, if a non-invasive sensing technique can be developed that requires the use of relatively small and light equipment and tools and whose cost is by far lower than that of each of other conventional non-invasive sensing techniques such as using ultrasound, it will be a breakthrough as a great technique which is very welcome by and pleasant to people.

### SUMMARY OF THE INVENTION

An objective of the present invention is to provide an apparatus or system for sensing a status of testicular torsion.

Another objective of the present invention is to provide a method for sensing a status of testicular torsion, using the apparatus of the present invention.

To achieve the foregoing objectives, in one aspect, the present invention provides an apparatus or system for sensing a status of testicular torsion in a test area (e.g. over a testicle) in a user, comprising: a sensor configured for projecting a specific wavelength light source to the test area and receiving a reflected light from the test area, for obtaining a pulse information and an oxygen saturation information (such as information of saturation of peripheral oxygen or SpO₂) on the test area; a comparing unit configured for determining a status of blood flow in the test area and a corresponding status of testicular torsion, according to the pulse information and the oxygen saturation information; and a display unit for displaying the testicular torsion status.

In one embodiment, the apparatus or system for sensing a status of testicular torsion may further comprise a second sensor configured for projecting a specific wavelength light source to the test area in the user and receiving a reflected light from the test area, in combination with the first sensor for obtaining a plurality of pulse information and a plurality of oxygen saturation information on the test area. The comparing unit is configured for determining the status of blood flow in the test area, by comparing the plurality of pulse information and the plurality of oxygen saturation information to each information type's reference value, and for determining a corresponding status of testicular torsion accordingly. And the sensors may be located on, in contact with, or covering the body skin over the organ under test.

In another embodiment, the apparatus or system for sensing a status of testicular torsion further comprises a second sensor for projecting the specific wavelength light source to a reference test area in the user and receiving a reflected light from the reference test area, for obtaining a second pulse information and a second oxygen saturation information; wherein the comparing unit is configured to compare the pulse information and the oxygen saturation information between the reference test area and the test area, for determining the status of blood flow in the test area as compared to a reference value on the reference test area, and also producing a relative pulse information and a relative oxygen saturation information on the test area. And a status of testicular torsion corresponding to the determined status of blood flow may then be determined. In this embodiment the reference test area and the test area may not overlap.

In various embodiments, the reference value or values of blood flow status may represent different blood flow statuses such as, without limitation, a smooth-flow status, a slightly-unsmooth-flow status, a mediumly-unsmooth-flow status, and a seriously-unsmooth-flow status. In various embodiments, the slightly-unsmooth-flow status may be due to e.g. a suspect testicular torsion, the mediumly-unsmooth-flow status may be due to e.g. a medium testicular torsion, and the seriously-unsmooth-flow status may be due to an emergency status (for example, a serious testicular torsion). In different embodiments, the blood flow status may be a lack-of-blood-flow status indicating the user's vascular blockage, testicular torsion, or lack of blood flow between his organs after surgery or during organ grafting or transplantation.

In one embodiment, the specific wavelength light source is of a specific wavelength susceptible to being absorbed by oxygenated hemoglobin (HbO₂) and deoxygenated hemoglobin (Hb) in human blood. In another embodiment, the specific wavelength light source comprises a red light and an infrared red light, and the comparing unit is configured for assessing the oxygen saturation information according to the reflected light of the red light and the infrared red light. And in another embodiment, the comparing unit is configured for assessing the oxygen saturation information according to the proportion in the reflected light between the red light and the infrared red light.

In one embodiment, the specific wavelength light source comprises a visible light source or an invisible light source, and the comparing unit is configured for assessing the pulse information according to the reflected light.

In one embodiment, the test area is located on the body skin or tissue over the organ under test where there are dense blood vessels. In an embodiment, the test area is over a testicle or scrotum of the user.

In another embodiment, the apparatus for sensing a status of testicular torsion further comprises an accelerometer for sensing the user's motion; and the accelerometer is configured to produce a warning when sensing an unusual motion of the user and configured to adjust the reference value (e.g. of said each information type or on the reference test area) when sensing a change in motion or posture of the user.

In another embodiment, the apparatus for sensing a status of testicular torsion further comprises a thermometer for measuring the temperature of the user or the apparatus; wherein the reference value (e.g. of said each information type or on the reference test area) is to be adjusted when the thermometer senses a change in the temperature; or the thermometer is configured to sense that the apparatus' operation temperature is so high as to warrant adjusting the frequency of performing said functions of the sensors, changing to sensing by the sensors to obtain either pulse information or oxygen saturation information on the test area for determining the status of blood flow in the test area, or reducing the number of the sensors used for performing their functions.

In one embodiment, the apparatus for sensing a status of testicular torsion further comprises protection pants, wherein the sensor is positioned in the protection pants corresponding to the test area, so that the sensor is capable of being maintained corresponding to the test area when the protection pants are being worn by the user.

The objectives, technical details, features, and effects of the present invention will be better understood with regard to the detailed description of the embodiments below, with reference to the drawings.

### BRIEF DESCRIPTION OF THE DRAWINGS

FIG. 1A shows an embodiment of an apparatus for sensing a status of testicular torsion according to the present invention.

FIG. 1B shows a second embodiment of an apparatus for sensing a status of testicular torsion according to the present invention.

FIG. 1C and FIG. 1D show operations of an oxygen saturation sensor according to the present invention.

FIG. 2 shows a third embodiment of an apparatus for sensing a status of testicular torsion according to the present invention.

FIG. 3 shows a flow chart of an embodiment of a method for sensing a status of testicular torsion according to the present invention.

FIG. 4 shows a fourth embodiment of an apparatus for sensing a status of testicular torsion according to the present invention.

### DESCRIPTION OF THE PREFERRED EMBODIMENTS

The present invention provides a designed application using a non-invasive light modulation technique. The technique may be applied to a test area of human body skin or epidermis covering the organ under test where there are dense blood vessels, by projecting two beams of different light source of specific wavelengths susceptible to being absorbed by oxygenated hemoglobin (HbO₂) and deoxygenated hemoglobin (Hb) in human blood to the test area covering the organ under test. The two beams include, for example, a red light (R, with a wavelength range from 620 nm to 770 nm) and an infrared red light (IR, with a wavelength range from 770 nm to 1000 nm). While the two beams are projected to the test area covering the organ under test, there employ the two beams' very different absorption characteristics in the spectrum between about 600 nm and 900 nm. That is to say, HbO₂ and Hb have very different absorptivities at the two light sources, such as, the absorptivity of Hb is higher than the absorptivity of HbO₂ at 600 nm for red light, while the absorptivity of HbO₂ is higher than the absorptivity of Hb at 900 nm for infrared red light. The fact that HbO₂ and Hb have very different absorptivities at the two light sources can be used for sensing or detecting optical properties of human tissues located about several millimeters to several centimeters under human body skin, to obtain or discover hemodynamic reactive states of the organ under test, including parameters such as SpO₂, erythrocyte saturation, and blood flow amounts.

According to the physical phenomenon that intensity of penetrating light varies with the concentration of each of oxygenated hemoglobin (HbO₂) and deoxygenated hemoglobin (Hb) in human blood, a signal of change in the concentration of each of oxygenated hemoglobin (HbO₂) and deoxygenated hemoglobin (Hb) in human blood can be obtained. Then a light-to-electricity transformation technique, such as photoelectric effect transformation technique, is used to obtain electrical signals of these two types of hemoglobins. Using calculations by a programmed microprocessor, oxygen saturation can be calculated according to a definitional formula of oxygen saturation. In a simple statement, red light and infrared red light can be used to detect oxygenated hemoglobin in human blood, how much of oxygenated hemoglobin is in human blood can be discovered or known by using the strength of penetrating or reflected light, and then oxygen saturation can be calculated using a formula.

In general, a change in SpO₂ is closely associated with and related to some degree to the blood flow status. Based on the principle of light modulation technique mentioned above, the invention is further designed to be applicable to sensing or detecting special blood flow statuses such as a testicular torsion status in human testicles. For example, a change in SpO₂ is closely associated with and related to some degree to each of the statuses of human testicular torsion, vascular blockage, bad blood circulation around a topical area or wound, and lack of blood flow between organs after surgery or during organ grafting or implantation. Therefore, the invention can be applied to accurately determine a status of blood flow in the above-mentioned areas or tissues therein and thereby to determine a (serious) status such as of testicular torsion.

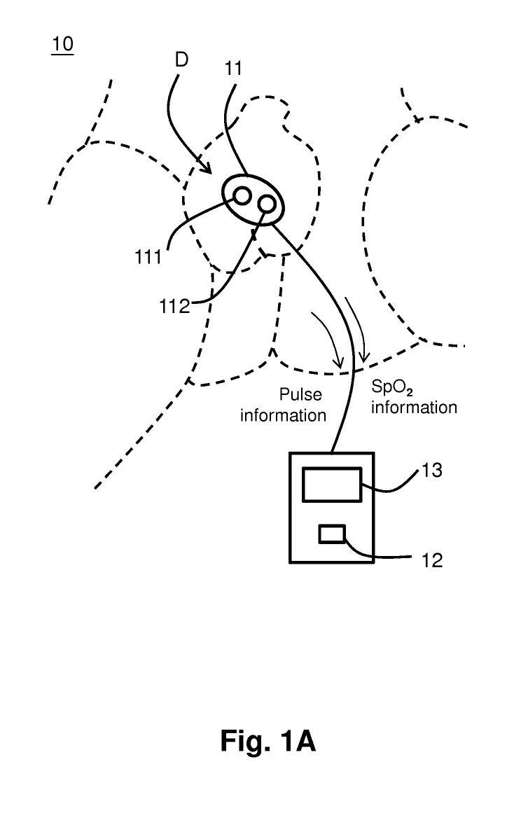

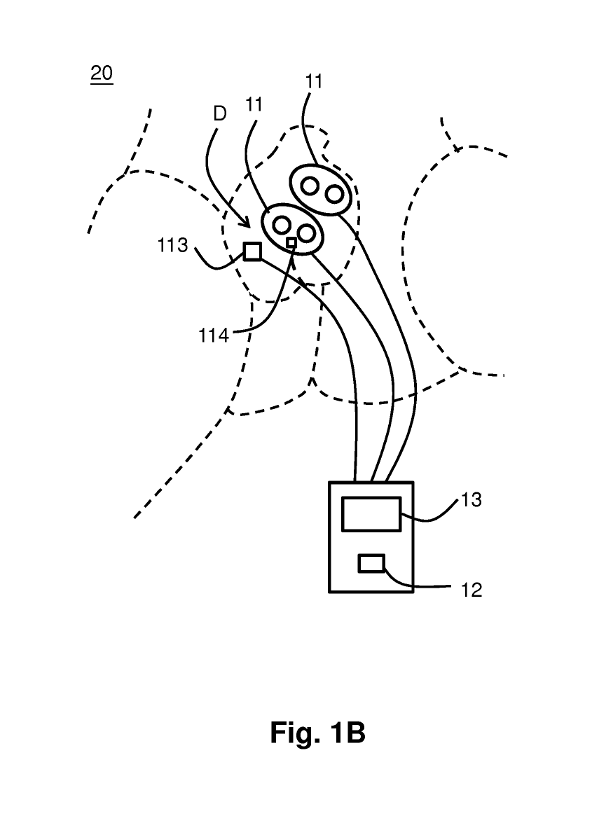

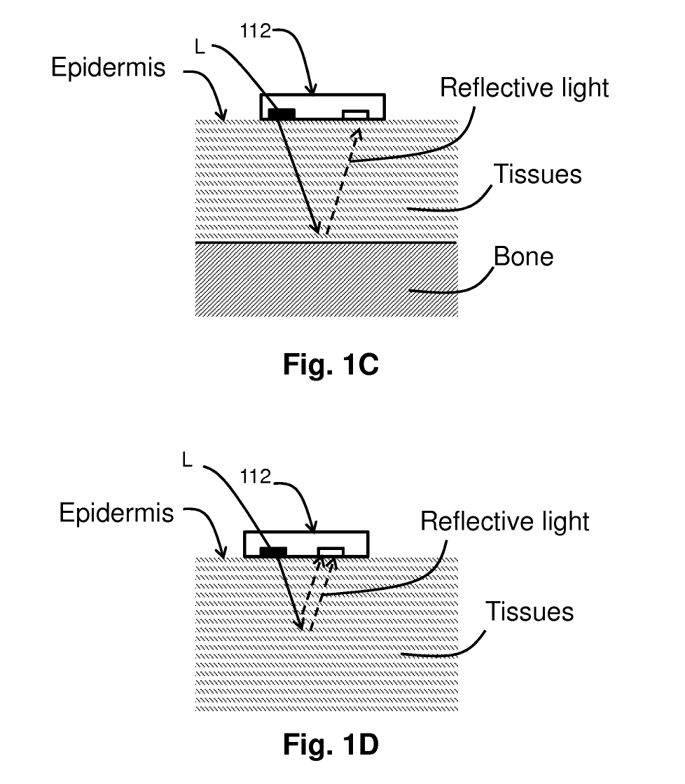

An apparatus for sensing a status of testicular torsion 10 and its operation method according to the invention are described as follows. The sensor 11 may initially project a light of specific wavelength, such as an infrared red light, to pass through the test area, and a receiver at an end of the sensor 11 is used to measure the absorbed amount of said light source. Referring to FIG. 1A, an embodiment of the present invention is an apparatus 10 for sensing a status of testicular torsion in a test area D of a user, which apparatus 10 includes a sensor 11 configured for projecting a specific wavelength light source to the test area D and receiving a reflected light from the test area D, for obtaining a pulse information and a SpO₂ information on the test area. The apparatus 10 also includes a comparing unit 12 configured for determining a status of blood flow in the test area D, as compared with a reference value (e.g. an index or factor of blood flow status or of each of the two information types), according to the pulse information and the SpO₂ information. The apparatus 10 also includes a display unit 13 configured for displaying the blood flow status. The location of the test area D, and a comparison result between the blood flow status and the reference value may be displayed by the display unit 13. In one embodiment, the sensor 11 may include a pulse information sensor 111 and a SpO₂ information sensor 112.

Since HbO₂ and Hb in human blood have different degrees of absorbing infrared red light, concentrations of the two kinds of hemoglobin or erythrocyte in human blood can be obtained by measuring their corresponding absorption degrees. Below is a formula for calculating the percentage reading of SpO₂ with the included terms' meanings:

Percentage reading of SpO₂=concentration of HbO₂÷(concentration of HbO₂+concentration of Hb)×100%

When the percentage reading of SpO₂ falls within a range between about 96% and 100%, such a reading of oxygen saturation information indicates or may represent a normal SpO₂. The invention may be used with defining such a reading of SpO₂ as representing the blood flow status being a smooth-flow or normal status. When the percentage reading of SpO₂ falls within a range between about 91% and 95%, such a reading of oxygen saturation information indicates or may represent a slight hypoxia in blood flow. The invention may be used with defining such a reading of SpO₂ as representing the blood flow status being a slightly-unsmooth-flow status. When the percentage reading of SpO₂ falls within a range between about 86% and 90%, such a reading of oxygen saturation information indicates or may represent a medium hypoxia in blood flow. The invention may be used with defining such a reading of SpO₂ as representing the blood flow status being a mediumly-unsmooth-flow status. When the percentage reading of SpO₂ is equal to or below about 85%, such a reading of oxygen saturation information indicates or may represent a serious hypoxia in blood flow. The invention may be used with defining such a reading of SpO₂ as representing the blood flow status being a seriously-unsmooth-flow status.

Referring to FIGS. 1C and 1D, it's known that human blood is composed basically of blood corpuscles or cells and blood plasma, and more than 98% of all corpuscles in blood are erythrocytes. The sensor 11 according to an embodiment of the invention may project a specific wavelength light source to the test area D and may sense by using the specific wavelength light as penetrating through and/or reflected from the test area D. The penetration mode of sensing is performed by projecting the specific wavelength light source L to the test area D, which projected light L then penetrates through the tissues (as to the bones) around the test area D and is reflected to outside of the epidermis to be received by the sensor 11. The penetration mode of sensing employs a deep tissue reflection, and is thus applicable to test or diagnosis areas such as in the fingers. The reflection mode of sensing is performed by projecting the specific wavelength light source L to the test area D, which projected light L permeates human organs in which there may be dense blood vessels and then is reflected to outside of the epidermis to be received by the sensor 11. Therefore, the reflection mode of sensing is applicable to test areas such as areas in the ears, testicles, kidney, and liver.

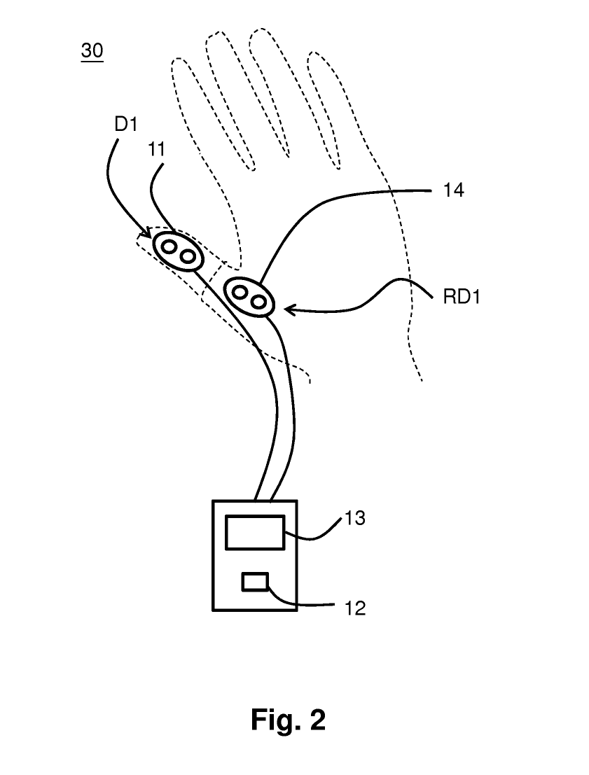

The test area D shown in FIG. 1A is located on the scrotum's epidermis over a testicle or the epididymis. The user can use the apparatus 10 for sensing a status of testicular torsion to sense or determine a status of blood flow in the test area D. When a lack of blood flow status occurs or is detected, the user can accordingly determine whether this is due to a testicular torsion. But the apparatus 10 is not limited to sensing a status of blood flow in a testicle, and it may be used to sense a status of blood flow around a topical area or wound. Referring to FIG. 2, in one embodiment, the apparatus 10 has the sensor 11 located on the test area D1 in a grafted organ. The apparatus 10 can be used to sense whether blood vessels in the grafted organ are connected with blood vessels in adjacent parts of the body. When they are connected, the pulse information and the SpO₂ information can be obtained by the apparatus 10 on the test area D1 in the grafted organ. Compared to sensing a status of the test area D, the reference value (e.g. of blood flow status or of each of the two information types) may be adjusted depending on different needs, for the apparatus 10 to sense a status of the test area D1. For example, to sense the status of weak blood flow connection in the test area D1 on the grafted organ, a lower reference value may be set to more easily detect and show the status of weak blood flow connection. Setting of the reference value will be explained with reference to the following description.

In addition, referring to FIG. 1B, another embodiment of the invention is shown. Compared with the embodiment shown in FIG. 1A, the apparatus 20 may include a plurality of sensors for sensing a status of the test area D to obtain a plurality of pulse information and a plurality of SpO₂ information. Two sensors 11 are shown in FIG. 1B as an example, but implementation of the invention is not limited to using two sensors, and three or more sensors may be used as long as purposes of the present invention are met. The comparing unit 12 is configured to determine a status of blood flow in the test area D by e.g. calculating an average among the plurality of pulse information and the plurality of SpO₂ information, or finding which one(s) of the plurality of pulse information and/or the plurality of SpO₂ information involves less noise, or calculating an average among those of the plurality of pulse information and/or the plurality of SpO₂ information that involve less noise. Less noise is involved when the pulse information can be clearly distinguished from other types of information (for example, respiratory information in a frequency spectrum close to that of the pulse information). When it is difficult to distinguish the pulse information from other types of information, more or not less noise is involved. And when they are clearly distinguished, less noise is involved. In another example, when one of the pulse information and the SpO₂ information is more distinguishable than the other, said one involves less noise.

In one embodiment, when sensing the strength or weakness of the blood flow is the focus of sensing, sensing to obtain SpO₂ information takes precedence over sensing to obtain the pulse information. Or when whether there is a substantial blood flow in the test area is the focus of sensing, one of the SpO₂ information and the pulse information which involves less noise may be chosen and then compared with the reference value. This is an exemplary illustration only and is in no way intended to limit the way of implementing the invention, which will depend on actual needs and circumstances.

In addition to the above mentioned different embodiments with different possible test areas, the system or apparatus for sensing a status of testicular torsion according to the invention may be used to sense a status of blood flow due to the user's vascular blockage, or a status of the user's vascular blockage. In this case, its way of implementation can be understood with reference to the rest of the description herein of other embodiments of the invention. And setting of the reference value in this case depends on actual needs.

In one embodiment, the specific wavelength light source is of a specific wavelength susceptible to being absorbed by oxygenated hemoglobin (HbO₂) and deoxygenated hemoglobin (Hb) in human blood. And the comparing unit is configured and used for assessing or obtaining the SpO₂ information (or the pulse information) according to the reflected light from the test area.

In another embodiment, the specific wavelength light source used with the invention comprises a red light and an infrared red light, and the comparing unit 12 is configured for assessing the SpO₂ information according to the proportion in the reflected light between the red light and the infrared red light. The comparing unit 12 can calculate the SpO₂ of a testicle according to the proportion in the reflected light between the red light and the infrared red light. Usually, the SpO₂ reading is between a range from 0% to 100%, and is collected with or supplemented by the reading of artery pulse. For example, the red light and the infrared red light are projected to a test area over a testicle, and then a photodetector is used to measure the proportion in the reflected light between the red light and the infrared red light, in order to obtain the pulse information and the SpO₂ information on the arteries in the testicle.

In various embodiments of the invention, the reference value or reference values of blood flow status may be defined to represent different blood flow statuses such as a smooth-flow status, a slightly-unsmooth-flow status, a mediumly-unsmooth-flow status, and a seriously-unsmooth-flow status. And the SpO₂ information (or the pulse information) can be compared with the reference value to determine or judge the blood flow status in the test area to be which of the smooth-flow status, the slightly-unsmooth-flow status, the mediumly-unsmooth-flow status, and the seriously-unsmooth-flow status. When the above-mentioned percentage reading of SpO₂ falls within a range between about 96% and 100%, such a reading of oxygen saturation information indicates or may represent a normal SpO₂, meaning the blood flow status being a smooth-flow or normal status. When the percentage reading of SpO₂ falls within a range between about 91% and 95%, such a reading of oxygen saturation information indicates or may represent a slight hypoxia in blood flow, meaning the blood flow status being a slightly-unsmooth-flow status. When the percentage reading of SpO₂ falls within a range between about 86% and 90%, such a reading of oxygen saturation information indicates or may represent a medium hypoxia in blood flow, meaning the blood flow status being a mediumly-unsmooth-flow status. When the percentage reading of SpO₂ is equal to or below about 85%, such a reading of oxygen saturation information indicates or may represent a serious hypoxia in blood flow, meaning the blood flow status being a seriously-unsmooth-flow status.

Generally speaking, the SpO₂ reading of a normal person will be higher than about 80%, and a person with insufficiency of respiratory functions will have a SpO₂ reading below about 80%. When a range of SpO₂ readings is close to 0%, such a range may represent that there is almost a lack of blood flow status on the test area. In this embodiment, a lack of blood flow status represents an emergency status requiring an emergency disposition or treatment.

In one embodiment, the reference value or reference values of blood flow status may be defined to represent different blood flow statuses such as a smooth-flow status, a slightly-unsmooth-flow status, a mediumly-unsmooth-flow status, and a seriously-unsmooth-flow status. And the SpO₂ information (or the pulse information) can be compared with the reference value to determine or judge the blood flow status on the test area to be which of the smooth-flow status, the slightly-unsmooth-flow status, the mediumly-unsmooth-flow status, and the seriously-unsmooth-flow status.

In one embodiment, the blood flow status may be a slightly-unsmooth-flow status between the normal flow status and the lack of blood flow status, which may represent a worsening from the normal flow status which needs a close monitoring. But the slightly-unsmooth-flow status may alternatively represent an improvement or gradual recovery from a worse blood flow status.

In one embodiment, the specific wavelength light source comprises a visible light source or an invisible light source, and the comparing unit is configured for assessing or determining the pulse information according to the reflected light.

In one embodiment, the test area is located on the body skin in the test area of the user in which there are dense blood vessels. The sensor can more easily sense to obtain the pulse information and the SpO₂ information on the test area in which there are dense blood vessels. But implementation of the invention is not limited to dense blood vessel areas. For example, when a visible light source is used in the invention to obtain the pulse information on the test area, the sensor may sense to obtain the pulse information according to the change in color or shape of the visible light reflected in the test area.

Referring to FIG. 2, the above discussed test area is not limited to covering a testicle or the scrotum of the user, and the invention may be used to sense a status of blood flow between organs after or during organ grafting or implantation. In one embodiment, the apparatus 30 for sensing a status of testicular torsion further comprises a sensor 14 for projecting the specific wavelength light source to a reference test area RD1 in the user and then receiving a reflected light from the reference test area RD1, for obtaining a second pulse information and a second SpO₂ information on the reference test area RD1. And the comparing unit 12 is configured to compare the pulse information and the oxygen saturation information between the reference test area RD1 and the test area D1, for determining the status of blood flow in the test area D1 as compared to a reference value on the reference test area RD1, and also producing a relative pulse information and a relative oxygen saturation information on the test area D1. The reference test area RD1 is for obtaining the second pulse information and the second SpO₂ information against which the apparatus 30 compares the pulse information and the SpO₂ information on the test area D1 in the same user. When the pulse information and SpO₂ information of a particular user deviates much from a general or normal user's such types of information (for example because of the particular user's respiratory disorder), directly (or in the first place) sensing a status of the test area D1 in the particular user is susceptible to resulting in a wrong determination of the status. The addition of sensing a status of the reference test area RD1 in the particular user helps maintain the accuracy of sensing by the apparatus 30 of a status of unusual or abnormal test areas in particular persons.

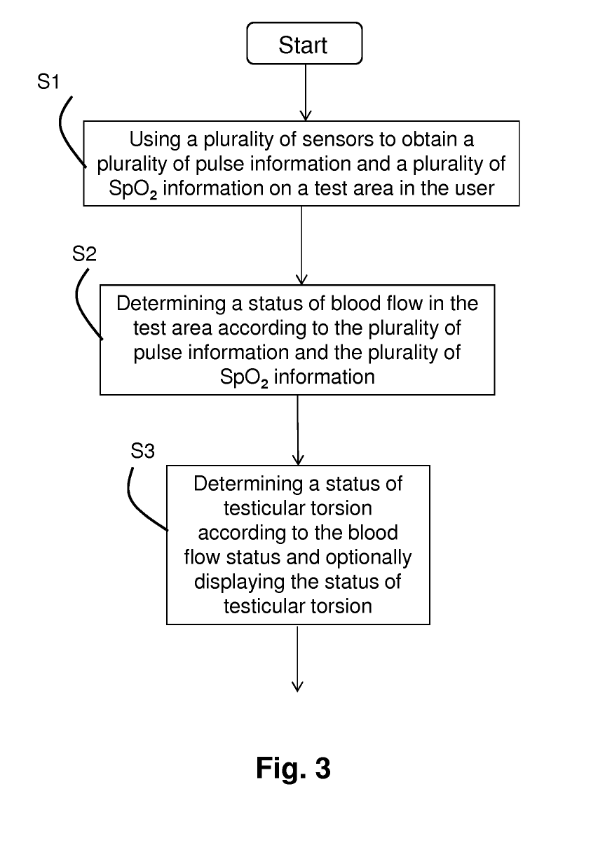

With reference to FIG. 3, the flow chart shows an embodiment of a method provided by the present invention for sensing a status of testicular torsion, which method comprises the following: using a plurality of sensors to obtain a plurality of pulse information and a plurality of SpO₂ information on a test area in the user (S1); determining a status of blood flow in the test area according to the plurality of pulse information and the plurality of SpO₂ information (S2); and determining a status of testicular torsion according to the blood flow status and optionally displaying the status of testicular torsion (S3). The sensors, pulse information, SpO₂ information, test area, and blood flow status have been illustrated in the above description of different embodiments of the invention.

The connections among the sensor(s), the comparing unit, and the display unit according to the invention are not limited to being implemented by cables or wires, and can also be implemented by wireless connections.

In addition, referring to FIG. 1B, the apparatus 20 may comprise an accelerometer 113 for sensing a change in the user's motion or posture, such as changing to lying flat, standing up from a sitting posture, or a change between standing and sitting by the user. When the user straightens up or raises (a part of) his body, his pulse strength will correspondingly increase, so the reference value should correspondingly be increased. In addition, when an unusual motion occurs (for example, when the user tears the sensor or the sensor falls on the ground), the apparatus 20 can use the accelerometer 113 to sense the unusual motion, in order to produce a warning informing related people for their coming to handle. The accelerometer 113 as shown in FIG. 1B is independent of other sensors, but the invention isn't so limited. Depending on actual needs, the accelerometer 113 may be connected or assembled with other sensors.

Continuing to refer to FIG. 1B, the apparatus 20 may further comprise a thermometer 114 for measuring the temperature of the user or the apparatus. The reference value discussed above may be adjusted when the thermometer 114 senses a change in the temperature. For example, when the temperature increases, the pulse strength will usually correspondingly decrease and the reference value may correspondingly be adjusted by being reduced, In another embodiment, the thermometer 114 may be configured to sense that the apparatus' operation temperature is so high as to warrant adjusting the frequency of performing said functions of the sensors, changing to sensing by the sensors to obtain either pulse information or oxygen saturation information on the test area D for determining the status of blood flow in the test area D, or reducing the number of the sensors used for performing their functions. These reactions will help lower the temperature of the apparatus 20.

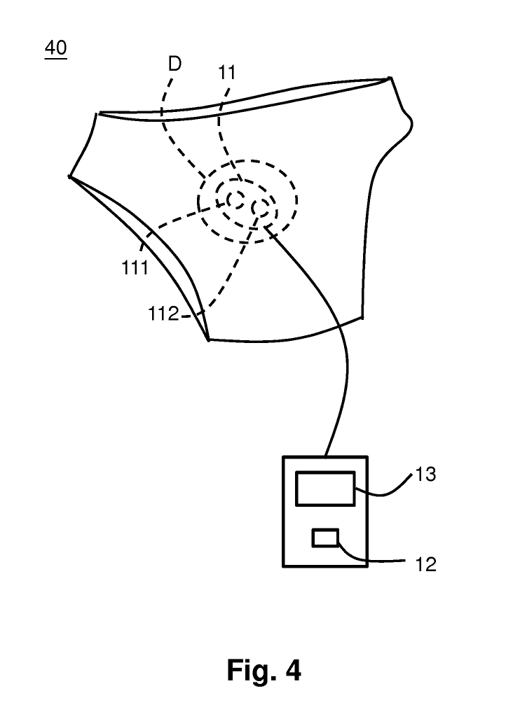

Referring to FIG. 4, the apparatus 20 may further comprise protection pants 115, and the sensor may be positioned or disposed in the protection pants corresponding to the test area. The sensor may be disposed in or on the protection pants by using a Velcro band, be designed to be put in an opening pocket of the protection pants, or be disposed using other design with similar functions. Therefore the sensors 111 and 112, (and maybe the thermometer 114 or the accelerometer 113) may be capable of being maintained corresponding to the test area when the protection pants 115 are being worn by the user.

The present invention has been described in considerable detail with reference to certain preferred embodiments thereof. It should be understood that the description is for illustrative purpose, not for limiting the scope of the present invention. Those skilled in this art can readily conceive variations and modifications within the spirit of the present invention.

What is claimed is:

1. An apparatus for sensing a status of testicular torsion in a test area in a user, the apparatus comprising:
a sensor configured for projecting a specific wavelength light source to the test area and receiving a reflected light from the test area, for obtaining a pulse information and an oxygen saturation information on the test area;
a comparing unit configured for determining a status of blood flow in the test area and a corresponding status of testicular torsion, according to the pulse information and the oxygen saturation information; and
a display unit for displaying the testicular torsion status.

2. The apparatus of claim 1, wherein the testicular torsion status is a no-torsion or normal status, a suspect-torsion status, a medium-torsion status, or a serious-torsion status.

3. The apparatus of claim 2, wherein the serious torsion status is due to a vascular blockage or a lack of blood flow between organs after surgery.

4. The apparatus of claim 3, wherein the test area is over a testicle or the scrotum of the user.

5. The apparatus of claim 1, wherein the blood flow status is a smooth-flow status, a slightly-unsmooth-flow status, a mediumly-unsmooth-flow status, or a seriously-unsmooth-flow status.

6. The apparatus of claim 5, wherein the blood flow status is a lack-of-blood-flow status due to a vascular blockage, a testicular torsion, or a lack of blood flow between organs after surgery or during organ grafting or transplantation.

7. The apparatus of claim 6, wherein when the test area is over a testicle or the scrotum of the user, the lack-of-blood-flow status is due to a serious testicular torsion.

8. The apparatus of claim 1, wherein the specific wavelength light source is of a specific wavelength susceptible to being absorbed by oxygenated hemoglobin (HbO₂) and deoxygenated hemoglobin (Hb) in human blood.

9. The apparatus of claim 1, wherein the specific wavelength light source comprises a visible light source or an invisible light source, and the comparing unit is configured for assessing the pulse information according to the reflected light.

10. The apparatus of claim 1, wherein the specific wavelength light source comprises a red light and an infrared red light, and the comparing unit is configured for assessing the oxygen saturation information according to the reflected light of the red light and the infrared red light.

11. The apparatus of claim 1, wherein the sensor is located on the body skin in the test area of the user in which there are dense blood vessels.

12. The apparatus of claim 1, further comprising a second sensor configured for projecting the specific wavelength light source to a reference test area in the user and receiving a reflected light from the reference test area, for obtaining a second pulse information and a second oxygen saturation information, wherein the reference test area and the test area don't overlap; wherein the comparing unit is configured to compare pulse information and oxygen saturation information between the reference test area and the test area, for determining the status of blood flow in the test area as compared to a reference value on the reference test area, and also producing a relative pulse information and a relative oxygen saturation information on the test area.

13. The apparatus of claim 12, further comprising an accelerometer for sensing the user's motion; wherein the accelerometer is configured to produce a warning when sensing an unusual motion of the user and configured to adjust the reference value when sensing a change in motion or posture of the user.

14. The apparatus of claim 12, further comprising a thermometer for measuring the temperature of the user or the apparatus; wherein the reference value is to be adjusted when the thermometer senses a change in the temperature; or the thermometer is configured to sense that the apparatus' operation temperature is so high as to warrant (1) adjusting the frequency of performing said functions of the sensors, (2) changing to sensing by the sensors to obtain either pulse information or oxygen saturation information on the test area for determining the status of blood flow in the test area, or (3) reducing the number of the sensors used for performing their functions.

15. The apparatus of claim 1 further comprising protection pants, wherein the sensor is positioned in the protection pants corresponding to the test area, so that the sensor is capable of being maintained corresponding to the test area when the protection pants are being worn by the user.

16. A method for sensing a status of testicular torsion in a test area in a user, using the apparatus of claim 4, comprising:
providing a first light projection apparatus capable of switching between a light emitting mode and a light reflecting mode;
using a sensor to project a specific wavelength light source to the test area;
receiving a reflected light from the test area upon the projecting step, to obtain a pulse information and an oxygen saturation information on the test area; and
determining a status of blood flow in the test area and a corresponding status of testicular torsion, according to the pulse information and the oxygen saturation information.

17. The method for sensing a status of testicular torsion according to claim 16, further comprising displaying the testicular torsion status on a display unit.
